(19) United States
(12) Patent Application Publication
(10) Pub. No.: US 2015/0085254 A1
(43) Pub. Date: Mar. 26, 2015

(54) Micro-Display Based Slit Lamp Illumination System

(71) Applicant: Topcon Medical Laser Systems, Inc., Santa Clara, CA (US)

(72) Inventors: Chris Sramek, Sunnyvale, CA (US); Greg Halstead, Sunnyvale, CA (US)

(21) Appl. No.: 14/037,969

(22) Filed: Sep. 26, 2013

(51) Int. Cl. A61B 3/00; A61B 3/10

(52) U.S. Cl. CPC A61B 3/0008; A61B 3/10
USPC 351/211; 351/246

(57) ABSTRACT

Methods and apparatuses for a micro-display based slit lamp illumination system are provided. A first optical element is configured to generate a micro-display image including an illuminated area. A second optical element is configured to receive the micro-display image, and focus the micro-display image upon an eye to be examined, wherein light is reflected from the eye as a result of the illuminated area.

### TECHNICAL FIELD

The present disclosure is generally directed to ophthalmic systems for use in diagnosing and treating conditions of the eye, and more specifically to illumination systems and methods for ophthalmic systems.

### BACKGROUND

A conventional slit lamp is an instrument consisting of a high-intensity light source. The high-intensity light source can be focused to shine a beam of light into a patient's eye. The beam of light is often focused to shine a desired light pattern into the patient's eye, such as a thin slit-shaped sheet of light.

Slit lamps are typically used in ophthalmic illumination systems to allow a practitioner to diagnose and treat conditions of the eye, e.g., by enabling a practitioner to view the patient's eye. For example, a slit lamp may be a component of a clinical bio-microscope used to facilitate an examination of structures within a patient's eye, including the eyelid, retina, sclera, conjunctiva, iris, lens and cornea.

A clinical bio-microscope is typically composed of a viewing system that is co-pivotal with a slit lamp to allow various angles of viewing and angles of illumination to a patient's eye. For example, a relatively oblique angle of illumination may be chosen to enhance the surface details and texture of a patient's eye by showing a shadowing on the distal edge of the subject. In contrast, a relatively direct coaxial angle of illumination may be chosen to more accurately show color, size and relative position of a subject (e.g., a retina) in relation to other anatomy. A relatively direct coaxial angle of illumination also may appear to flatten structures that would otherwise appear to be more three-dimensional when illuminated at a relatively severe angle.

Several factors can affect the quality of eye visualization, including opaque and highly reflective cornea tissue, iris color and other biological variables. As such, conventional slit lamps typically include orientation and angle settings (e.g., settings for various slit sizes and shapes), a rotating filter wheel (also known as a color wheel filter), and other mechanisms to allow for exposure adjustment control in an illuminated image of a patient's eye. In many existing ophthalmic illumination systems, however, slit lamp adjustment controls are limited, which can reduce the achievable quality of an illuminated image of a patient's eye that can be viewed by a practitioner or photographed.

### SUMMARY

A micro-display based slit lamp illumination system is provided. A first optical element is configured to generate a micro-display image including an illuminated area. A second optical element is configured to receive the micro-display image, and focus the micro-display image upon an eye to be examined, wherein light is reflected from the eye as a result of the illuminated area. The first optical element may be a micro-display projector and include one of a liquid crystal on silicon (LCoS), digital-micro-mirror (DMD) or micro-electro-mechanical systems (MEMS) micro-scanner and one of a visible (RGB) light-emitting diode (LED) or laser light source or invisible (infrared, ultraviolet) LED or laser light source.

In accordance with an embodiment, a controller may be configured to receive a parameter for generating the micro-display image, wherein the parameter is related to one of a color, shape or size of the illuminated area. The controller may transmit a command based on the parameter to the first optical element.

In accordance with an embodiment, the illuminated area may be one of a slit-shaped, round or polygonal-shaped area, and the micro-display image may include a plurality of illuminated areas.

In accordance with an embodiment, the micro-display image may include concurrent information. The concurrent information may relate to measurement information, patient data, a treatment parameter, a preoperative image or a treatment plan.

These and other advantages of the invention will be apparent to those of ordinary skill in the art by reference to the following detailed description and the accompanying drawings.

### BRIEF DESCRIPTION OF THE DRAWINGS

FIG. 1 shows a conventional slit-lamp system;

FIG. 2 shows a micro-display based slit-lamp illumination system in accordance with an embodiment;

FIG. 3 shows an image generated by a micro-display projector onto a patient's eye in accordance with an embodiment;

FIG. 4 shows an image generated by a micro-display based slit-lamp illumination system in accordance with an embodiment;

FIG. 5 shows an image generated by a micro-display based slit-lamp illumination system in accordance with an embodiment;

FIG. 6 shows an image generated by a micro-display based slit-lamp illumination system in accordance with an embodiment;

FIG. 7 shows an image generated by a micro-display based slit-lamp illumination system in accordance with an embodiment;

FIG. 8 shows an image generated by a micro-display based slit-lamp illumination system in accordance with an embodiment;

FIG. 9 is a flowchart of a method of micro-display based slit lamp illumination in accordance with an embodiment;

FIG. 10 is a high-level block diagram of an exemplary computer that may be used for the various embodiments herein.

### DETAILED DESCRIPTION

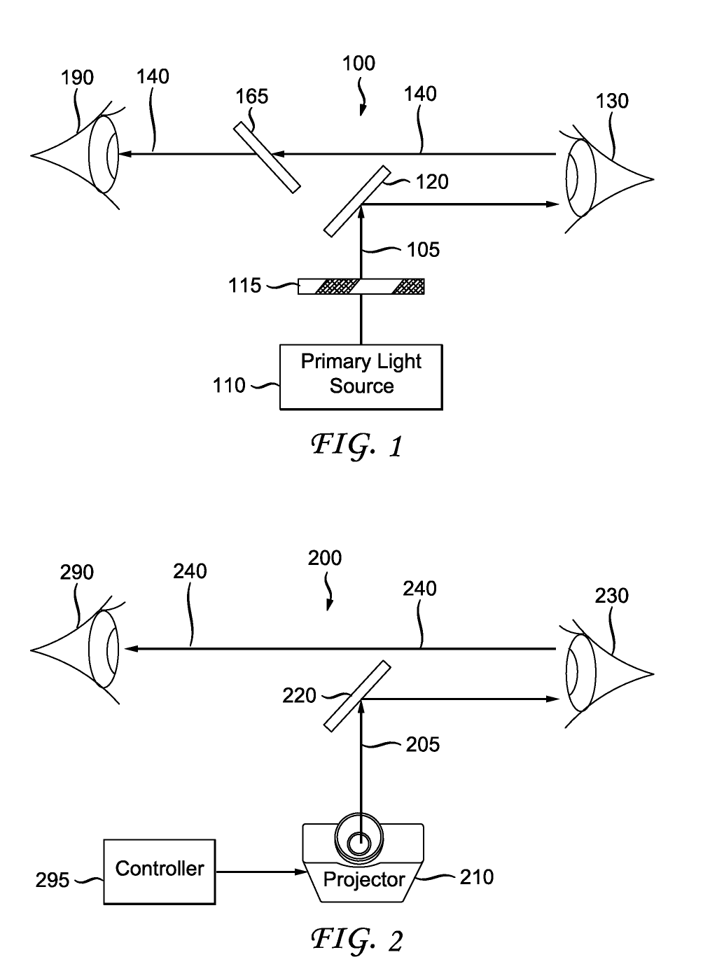

FIG. 1 shows a conventional slit-lamp system. A conventional slit-lamp illumination system typically includes a halogen lamp or white LED light source, a slit adjustment mechanism, optical relay, filter wheel, slit rotation prism assembly and exit (turning) prism/mirror. For example, system 100 comprises a primary light source 110, a mirror 120 and a beam splitter 165. Primary light source 110 generates light 105 which is directed via color wheel filter 115 and mirror 120 toward a patient's eye 130. The light strikes eye 130 and is reflected, generating reflected light 140. Reflected light 140 passes through beam splitter 165 and propagates toward a practitioner's eye 190, allowing the practitioner to view structures within patient's eye 130. Beam splitter 165 is typically adapted to allow a significant amount of reflected light 140 from patient's eye 130 to pass through depending on the application.

Primary light source 110 comprises a conventional slit lamp. Many conventional slit-lamp-based illumination systems use a high-intensity/high-pressure light source, such as a halogen light source that produces and channels white light to the slit lamp. The use of white light does not permit a practitioner to control with precision the color of the light that enters the slit lamp, and therefore limits the range of observations that can be made by the practitioner. As such, it is sometimes advantageous to observe certain structures of the eye, and/or certain medical conditions, using selected colors of light.

Typical illumination systems use one or more color filters to control the color of light delivered to the eye in order to facilitate the observation of certain aspects of the eye that may be difficult to visualize under white light. For example, color wheel filter 115 may be used to produce red, blue, or green light, to remove infrared light, or to otherwise select the color of light 105 that passes through to mirror 120. However, even with the use of filters, such as color wheel filter 115, the practitioner is limited by the filters currently available and therefore may not be able to achieve a desired level of precision in the selection of the color of light used.

FIG. 2 shows a micro-display based slit lamp illumination system in accordance with an embodiment. System 200 comprises a micro-display projector 210 and a mirror 220. In an embodiment, micro-display projector 210 is used in place of a conventional slit-lamp illumination assembly having a primary light source, such as primary light source 110 in FIG. 1.

In system 200, micro-display projector 210 generates a micro-display image 205 including an illuminated area which is directed by mirror 220 toward a patient's eye 230. Micro-display image 205 is displayed upon patient's eye 230 and is reflected at least in part based on the illuminated area, generating reflected light 240. Reflected light 240 propagates toward a practitioner's eye 290, allowing the practitioner to view structures within patient's eye 230.

Micro-display projector 210 may be any type of micro-display or pico projector comprising an optical engine (e.g., an illumination source, modulator and projection optics). For example, micro-display projector 210 may be a stand-alone projector or a projector that is integrated into another device, such as a mobile device (e.g., a mobile phone) or a notebook computer.

Micro-display projector 210 may include one of a liquid crystal on silicon (LCoS), digital-micro-mirror device (DMD), 2-D micro-electro-mechanical systems (MEMS) or 2-D X/Y galvanometer set micro-scanner for generating an image. Micro-display projector 210 also may comprise relay optics (e.g., to illuminate a micro-display with an illumination area dimension matching the micro-display size), and a collimation or projection lens.

Further, micro-display projector 210 may include one or more sources of visible and/or invisible illumination to be operable to form, e.g., an infrared or color image projection. The one or more sources of visible and/or invisible illumination may include a halogen lamp, a white light emitting diode (LED), one or more coaxial LEDs (e.g., red, green, blue, amber or near-infrared LEDs) or one or more coaxial lasers (e.g., red-green-blue (RGB) or near-infrared lasers). In an embodiment, an exemplary light source for micro-display projector 210 may have an illumination range of around 10-200 lumens. One skilled in the art will note that micro-display projector 210 may include several other elements, and that the micro-display projector features and components discussed herein are merely illustrative and, therefore, are not intended to be exhaustive.

In an embodiment, micro-display projector 210 generates micro-display projection 205 such that an image including an illuminated area is directed by mirror 220 for display upon patient's eye 230. For example, micro-display projector 210 may generate micro-display projection 205 to project one or more slit-shaped, round or polygonal-shaped areas or channels of white or colored light upon patient's eye 230. As such, micro-display projector 210 can be configured, e.g., via a command received from controller 295, to generate micro-display projections that allow for a wide range of observations to be made by a practitioner.

In an embodiment, controller 295 may be configured to receive user inputs via control switches, knobs, or a GUI interface (e.g. a touch-screen display or LCD with a mouse/trackpad interface), and transmit one or more commands to micro-display projector 210 to generate a micro-display projection 205 based on the one or more received user inputs. Controller 295 also may transmit one or more commands to micro-display projector 210 to adjust the color, brightness and timing of micro-display projection 205 based on one or more user inputs. Controller 295 also may be configured to receive inputs from one or more external sources (e.g. a camera flash trigger or a computer processing real-time slit-lamp video) and transmit commands to projector 210.

As such, micro-display projector 210 can generate a micro-display image 205 including illuminated areas having selected colors of light, thereby emulating the effect of color wheel filter 115, shown in FIG. 1. For example, micro-display image 205 may include an illuminated area of red, blue, green, infrared or ultraviolet light. However, unlike color wheel filter 115, micro-display projector 210 can be configured to generate micro-display projection 205 to achieve desired levels of precision in the selection of the color (e.g., color gradation) and intensity of light used.

In addition, micro-display projector 210 may be configured to emulate the operation of a conventional slit-lamp-based illumination system by allowing for various angles of viewing and angles of illumination to patient's eye 230. For example, micro-display projector 210 may be configured to swivel about an image plane or to scan the micro-display projection 205 of an image across a desired range (e.g., across a 180 deg range).

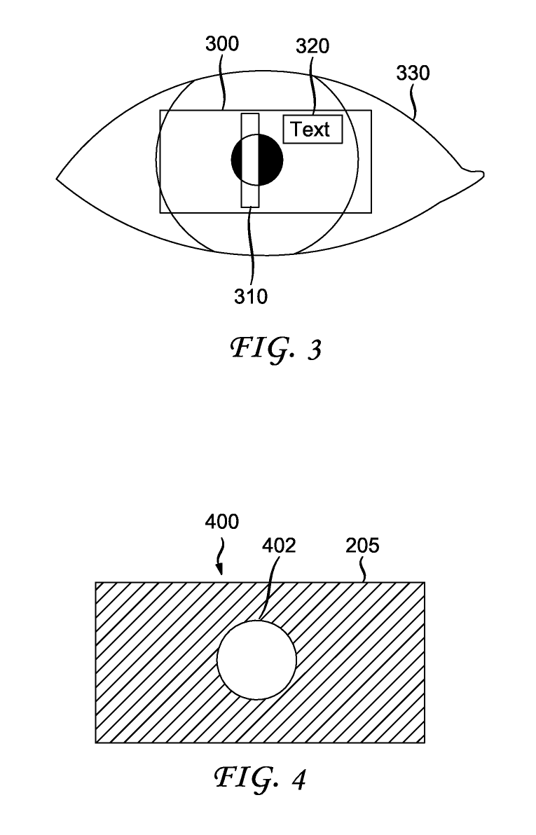

FIG. 3 shows an image generated by a micro-display projector onto a patient's eye in accordance with an embodiment. Micro-display image 300 includes an illuminated (slit-shaped) area 310 generated by micro-display projector 210 and directed by mirror 220 (shown in FIG. 2) onto patient's eye 330. In an embodiment, micro-display projector 210 also may generate concurrent information 320 that is integrated into micro-display image 300. Alternatively, all or part of concurrent information 320 may be received from a source external to micro-display projector 210 (e.g., from controller 295, or a source other than controller 295).

For example, concurrent information 320 may include visual information received or generated by micro-display projector 210, including any type of image or data that may be projected onto a patient's eye 330. Concurrent information 320 may include patient information, the current time and date, or other information that may be of use in a clinical environment. In another example, concurrent information 320 may measurement information regarding micro-display image 300, such as a measurement axis, distance, area, scale or grid. Measurement information also may include a current illumination area diameter, current slit width, inter-slit spacing, current filter choice, micrometer scale labeling, or circle/ellipse radii, ratios and areas.

When illumination system 200 is used in conjunction with therapy systems including laser systems and other equipment, concurrent information 320 may include one of a treatment parameter or a preoperative image, treatment plan, an aiming beam pattern or a treatment beam target indicator. For example, concurrent information 320 may be received from a laser system console to include information regarding treatment laser parameters, such as, e.g., power, spot-size and spacing for display as part of micro-display projection 300.

In accordance with various embodiments, micro-display projector 210 and controller 295 may be configured to create images corresponding to clinically useful slit-lamp settings, such as those shown in FIGS. 4-8 and described below. Micro-display projector 210 and controller 295 also may be configured to create images corresponding to any combination of the slit-lamp settings shown in FIGS. 4-8. For example, controller 295 may receive a parameter for generating a micro-display projection of an image having an illuminated area, wherein the parameter is related to one of a color, shape or size of the illuminated area, and transmit a command based on the parameter to micro-display projector 210.

FIG. 4 shows an image generated by a micro-display based slit lamp illumination system in accordance with an embodiment. Image 400 illustrates a micro-display image 205 including an illuminated circular-shaped area 402. For example, area 402 may be between 0.2 mm to >=8 mm in diameter (e.g., based on a 20 mm maximum for a typical eye surface area). In an embodiment, the diameter of area 402 may be continuously adjustable, e.g., in response to commands transmitted from controller 295 to micro-display projector 210. In another embodiment, area 402 may be user-adjustable based on color, including white (unfiltered), blue (“cobalt blue”), green (red-free), 10% intensity (grey) or other illumination settings of micro-display projector 210. For example, a user may have discrete control of each color channel of area 402.

As such, at controller 295 color gradation may be selectable via preset red-green-blue (RGB) intensity settings or may be continuously variable based on user inputs.

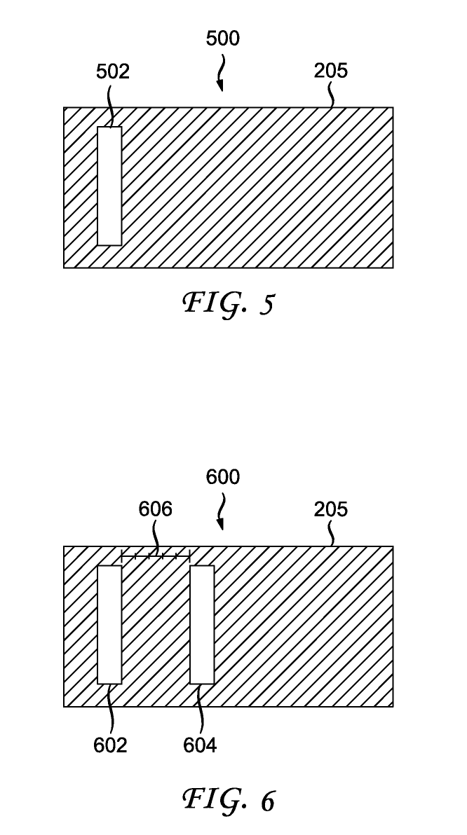

FIG. 5 shows an image generated by a micro-display based slit lamp illumination system in accordance with an embodiment. Image 500 illustrates a micro-display image 205 including an illuminated slit-shaped area 502. For example, area 502 may be between 0.2 mm to >=8 mm in length (e.g., based on a 20 mm maximum for a typical eye surface area), continuously adjustable between 0 mm to up to >=8 mm in width (20 mm maximum) and continuously adjustable (e.g., +/−90 deg) in orientation. As such, the slit may be centered (coaxial) or offset within a coaxial field-of-view.

FIG. 6 shows an image generated by a micro-display based slit lamp illumination system in accordance with an embodiment. Image 600 illustrates a micro-display image 205 including illuminated double slit-shaped areas 602 and 604. Double slit-shaped areas 602 and 604 may have adjustable parameters similar to those of area 502 above. For example, areas 602 and 604 each may be between 0.2 mm to >=8 mm in length (e.g., based on a 20 mm maximum for a typical eye surface area), continuously adjustable between 0 mm to up to >=8 mm in width (20 mm maximum) or continuously adjustable (e.g., +/−90 deg) in orientation. In addition, areas 602 and 604 may be adjustable with regard to inter-slit spacing, e.g., from 0 mm up to >=8 mm (e.g., for a 20 mm maximum area width). For example, image 600 may include concurrent information 606 related to real-time spacing offset information (i.e., measurement information) with regard to inter-slit spacing.

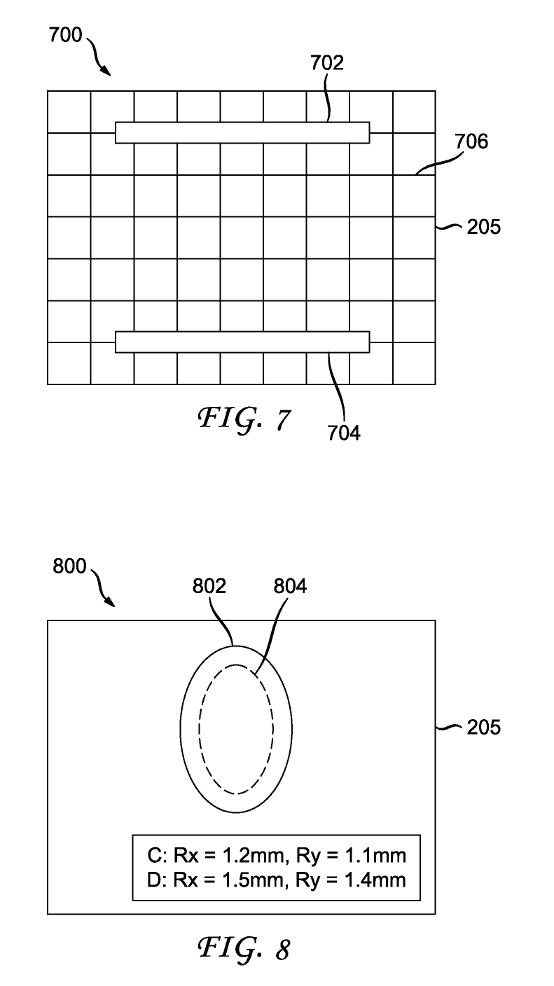

FIG. 7 shows an image generated by a micro-display based slit lamp illumination system in accordance with an embodiment. Image 700 illustrates a micro-display image 205 including micrometer (e.g., reticle) areas 702 and 704 and grid 706, wherein the scale size, major and minor units of the areas are adjustable. For example, areas 702 and 704 and grid 706 may be useful for various clinical measurements, including pupil diameter, anterior chamber angle depth (non-gonioscopic), depth of foreign-bodies in cornea, gonioscopic measurement of iridocorneal angles, measurement of tear film meniscus height and rim tissue width around an optic nerve head.

FIG. 8 shows an image generated by a micro-display based slit lamp illumination system in accordance with an embodiment. Image 800 illustrates a micro-display image 205 including circle/ellipse contours 802 and 804 and concurrent information 806 related to major and minor radii of contours 802 and 804. For example, major and minor radii may be adjusted (e.g., via controller 295) for measuring pupil diameter or a cup-to-disc ratio of optic nerve head. Contours 802 and 804 and concurrent information 806 may be useful for various clinical measurements, including pupil diameter, anterior chamber angle depth (non-gonioscopic), depth of foreign-bodies in cornea, gonioscopic measurement of iridocorneal angles, measurement of tear film meniscus height and rim tissue width around an optic nerve head.

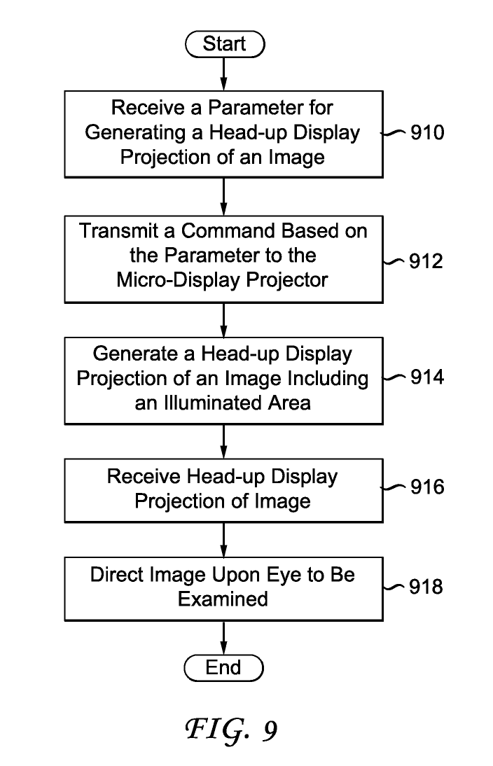

FIG. 9 is a flowchart of a method of micro-display based slit lamp illumination in accordance with an embodiment. FIG. 9 is discussed below with reference also to FIG. 2.

At step 910, a parameter for generating the micro-display image is received. Referring to FIG. 2, controller 295 may be configured to receive a parameter for generating the micro-display image, wherein the parameter is related to one of a color, shape or size of the illuminated slit image. In an embodiment, controller 295 may be configured to receive a parameter for generating the micro-display image to further include concurrent information relating to patient data, a treatment parameter, a preoperative image, or a treatment plan.

At step 912, a command based on the parameter is transmitted to micro-display projector 210. Referring to FIG. 2, controller 295 transmits a command based on the parameter to micro-display projector 210, wherein micro-display projector 210 generates micro-display image 205 in accordance with the command.

At step 914, a first optical element is configured to generate an image including an illuminated area. For example, the first optical element may be a micro-display projector including one of a liquid crystal on silicon (LCoS), digital-micro-mirror (DMD) or micro-electro-mechanical systems (MEMS) micro-scanner and one of a light-emitting diode (LED) or red-green-blue (RGB) laser light source. Referring to FIG. 2, micro-display projector 210 generates micro-display image 205 including an illuminated area (e.g., in accordance with the command received from controller 295). For example, the illuminated area may be a slit-shaped, round or polygonal-shaped area.

At step 916, a second optical element is configured to receive the micro-display image. Referring to FIG. 2, mirror 220 receives micro-display image 205 generated by micro-display projector 210.

At step 918, the second element is configured to direct the projection of the image upon an eye to be examined, wherein light is reflected from the eye as a result of the image. Referring to FIG. 2, mirror 220 directs the micro-display image toward a patient's eye 230. Micro-display image 205 is displayed upon eye 230, and the image is reflected, generating reflected light 240 which propagates toward a practitioner's eye 290, allowing the practitioner to view structures within the patient's eye 230. For example, the reflected light may include an image of structures within patient's eye 230 due to an illuminated area of micro-display image 205.

As such, a micro-display slit-lamp illumination system as disclosed herein may serve as a replacement for a traditional slit-lamp illuminator. Moreover, the micro-display slit-lamp illumination system can extend the capabilities of a traditional slit-lamp illuminator from simple illumination to quantification of observed tissue, as well as presentation of additional clinically relevant information.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be used within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the method steps described herein, including one or more of the steps of FIG. 9. Certain steps of the methods described herein, including one or more of the steps of FIG. 9, may be performed by a server or by another processor in a network-based cloud-computing system. Certain steps of the methods described herein, including one or more of the steps of FIG. 9, may be performed by a client computer in a network-based cloud computing system. The steps of the methods described herein, including one or more of the steps of FIG. 9, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of FIG. 9, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

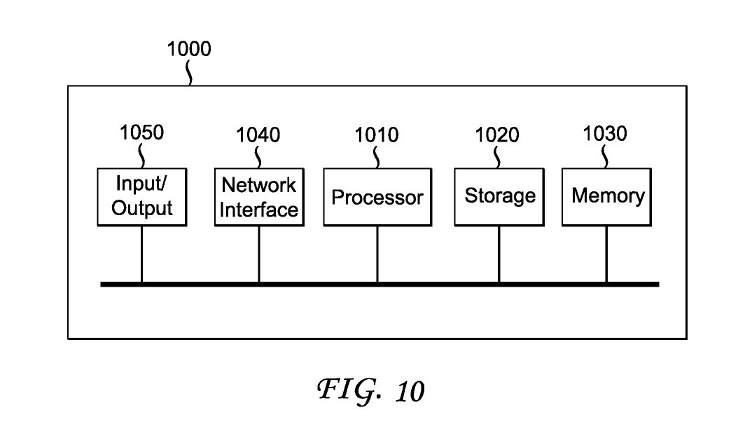

A high-level block diagram of an exemplary computer that may be used to implement systems, apparatus and methods described herein is illustrated in FIG. 10. Computer 1000 comprises a processor 1010 operatively coupled to a data storage device 1020 and a memory 1030. Processor 1010 controls the overall operation of computer 1100 by executing computer program instructions that define such operations. The computer program instructions may be stored in data storage device 1020, or other computer readable medium, and loaded into memory 1030 when execution of the computer program instructions is desired. Thus, the method steps of FIG. 9 can be defined by the computer program instructions stored in memory 1030 and/or data storage device 1020 and controlled by processor 1010 executing the computer program instructions. For example, the computer program instructions can be implemented as computer executable code programmed by one skilled in the art to perform an algorithm defined by the method steps of FIG. 9. Accordingly, by executing the computer program instructions, the processor 1010 executes an algorithm defined by the method steps of FIG. 9. Computer 1000 also includes one or more network interfaces 1040 for communicating with other devices via a network. Computer 1000 also includes one or more input/output devices 1050 that enable user interaction with computer 1000 (e.g., display, keyboard, mouse, speakers, buttons, etc.).

Processor 1010 may include both general and special purpose microprocessors, and may be the sole processor or one of multiple processors of computer 1000. Processor 1010 may comprise one or more central processing units (CPUs), for example. Processor 1010, data storage device 1020, and/or memory 1030 may include, be supplemented by, or incorporated in, one or more application-specific integrated circuits (ASICs) and/or one or more field programmable gate arrays (FPGAs).

Data storage device 1020 and memory 1030 each comprise a tangible non-transitory computer readable storage medium. Data storage device 1020, and memory 1030, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1050 may include peripherals, such as a printer, scanner, display screen, etc. For example, input/output devices 1150 may include a display device such as a cathode ray tube (CRT), plasma or liquid crystal display (LCD) monitor for displaying information to the user, a keyboard, and a pointing device such as a mouse or a trackball by which the user can provide input to computer 1100.

Any or all of the systems and apparatus discussed herein, including micro-display projector 210 and controller 295 may be implemented using a computer such as computer 1000.

One skilled in the art will recognize that an implementation of an actual computer or computer system may have other structures and may contain other components as well, and that FIG. 10 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

We claim:

1. A micro-display based slit lamp illumination system, comprising:
a first optical element configured to generate a micro-display image including an illuminated area; and
a second optical element configured to:
receive the micro-display image, and
direct the micro-display image upon an eye to be examined.

2. The system of claim 1, further comprising a controller configured to:
receive a parameter for generating the micro-display image, wherein the parameter is related to one of a color, shape or size of the illuminated area; and
transmit a command based on the parameter to the first element.

3. The system of claim 1, wherein the illuminated area is one of a slit-shaped, round or polygonal-shaped area.

4. The system of claim 1, wherein the image includes a plurality of illuminated areas.

5. The system of claim 1, wherein the micro-display image includes concurrent information.

6. The system of claim 5, wherein the concurrent information relates to measurement information, patient data, a treatment parameter, a preoperative image or a treatment plan.

7. The system of claim 1, wherein the first optical element includes one of a liquid crystal on silicon (LCoS), digital-micro-mirror (DMD) or micro-electro-mechanical systems (MEMS) micro-scanner.

8. The system of claim 1, wherein the first optical element includes one of a visible laser or light-emitting diode (LED) light source or an invisible laser or LED light source.

9. The system of claim 1, wherein the first optical element is a micro-display projector.

10. A micro-display based slit lamp illumination method, comprising:
generating a micro-display image including an illuminated area; and
transmitting the micro-display image to be focused upon an eye to be examined, wherein light is reflected from the eye as a result of the illuminated area.

11. The method of claim 10, further comprising:
receiving a parameter for generating the micro-display image, wherein the parameter is related to one of a color, shape or size of the illuminated area; and
transmitting a command based on the parameter to the first optical element.

12. The method of claim 10, wherein the illuminated area is one of a slit-shaped, round or polygonal-shaped area.

13. The method of claim 10, wherein the micro-display image includes a plurality of illuminated areas.

14. The method of claim 10, wherein the micro-display image includes concurrent information.

15. The method of claim 14, wherein the concurrent information relates to measurement information, patient data, a treatment parameter, a preoperative image or a treatment plan.

16. A micro-display based slit lamp illumination system, comprising:
a micro-display projector configured to generate a micro-display image including an illuminated area; and
a mirror configured to:
receive the projection of the micro-display image, and
direct the projection of the micro-display image upon an eye to be examined.

17. The system of claim 16, further comprising a controller configured to:
receive a parameter for generating the micro-display image, wherein the parameter is related to one of a color, shape or size of the illuminated area; and
transmit a command based on the parameter to the first optical element.

18. The system of claim 16, wherein the illuminated area is one of a slit-shaped, round or polygonal-shaped area.

19. The system of claim 16, wherein the micro-display image includes a plurality of illuminated areas.

20. The system of claim 16, wherein the micro-display image includes concurrent information.

21. The system of claim 20, wherein the concurrent information relates to measurement information, patient data, a treatment parameter, a preoperative image or a treatment plan.

22. The system of claim 16, wherein the micro-display projector includes one of a liquid crystal on silicon (LCoS), digital-micro-mirror (DMD) or micro-electro-mechanical systems (MEMS) micro-scanner.

23. The system of claim 16, wherein the micro-display projector includes one of a visible laser or light-emitting diode (LED) light source or an invisible laser or LED light source.
